(19) United States
(12) Patent Application Publication
(10) Pub. No.: US 2004/0121399 A1
(43) Pub. Date: Jun. 24, 2004

(54) Substrate bound linker molecules for the construction of biomolecule microarrays

(75) Inventors: Phillip J. Brock, Sunnyvale, CA (US); Richard A. Dipietro, Campbell, CA (US); Nicolette S. Fender, San Jose, CA (US); Robert D. Miller, San Jose, CA (US); Sally A. Swanson, San Jose, CA (US); Gregory M. Wallraff, Morgan Hill, CA (US)

Correspondence Address: SCULLY SCOTT MURPHY & PRESSER, PC, 400 GARDEN CITY PLAZA, GARDEN CITY, NY, 11530

(73) Assignee: International Business Machines Corporation, Armonk, NY

(21) Appl. No.: 10/324,189

(22) Filed: Dec. 20, 2002

(51) Int. Cl. G01N 33/53; G01N 31/00; G01N 33/543

(52) U.S. Cl. 435/7.1; 436/518

(57) ABSTRACT

A series of photoactivatible surface bound linker molecules, which can be used to fabricate biomolecular arrays, is described. Specifically, a composition which includes a solid substrate; an organic linking group having one terminal end portion bound to the solid substrate and at least one other terminal end portion containing an alcohol or carbonyl functionality; and an acid labile protecting group selected from acetals and ketals bound to the alcohol or carbonyl functionality. A composition which comprises a solid substrate; an organic linking group having one terminal end portion bound to the solid substrate and at least one other terminal end portion containing an aldehyde group is also described. The present invention further provides a composition which includes a solid substrate; and at least one of a photoacid generator or a sensitizer bound to the solid substrate.

### FIELD OF THE INVENTION

The present invention relates to biomolecular arrays, and more particularly to a series of photoactivatible surface bound linker molecules that can be used to fabricate biomolecular arrays using efficient, high yield techniques.

### BACKGROUND OF THE INVENTION

The fabrication and use of microarrays of nucleotides, so called “DNA chips” disclosed, for example, in E. S. Lander, Nature Genetic Supplement 1999, 21, 3 and arrays of substrate bound proteins disclosed, for example, in G. Macbeth, et al., Science 2001, 289, 1760 have become areas of intense interest following the recent publication of the first draft of the human genome (see, for example, Science 2001, 291, 1145-1134). Microarrays of oligonucleotides and cDNA's can be fabricated using a variety of techniques including robot spotting, ink jet printing and in the case of oligonucleotides, lithographic in-situ synthesis. Some of these prior art methods can be applied to the production of chips as well.

All of the above mentioned prior art methods rely on covalent attachment of the nucleic acids or polypeptides to some sort of solid support in a patternwise fashion either via a physical deposition or optical delineation. While these various ‘first generation’ methods are being used to fabricate low quantities of microarrays useful in research purposes, large scale implementation of these diagnostic devices will require high volume, efficient manufacturing methods that preferably can be used to fabricate both DNA and protein arrays.

G. Wallraff, et al., Proc. Natl. Acad. Sci. 1996, 93, 13555 describe a technique for the lithographic fabrication of oligonucleotide arrays based on photoacid chemistry used in chemically amplified resists. This prior art approach, which is termed “Photoacid Patterned Array (PPA)” has a number of potential advantages over the photoremovable protecting groups method disclosed, for example, in S. P. A. Fodor, et al., Science 1991, 251, 767 currently employed by Affymetrix in the manufacture of Gene Chips™. The PPA approach is based on standard nucleic acid synthesis chemistry (see FIG. 1) where the acid catalyst is generated in the patterned polymer film overcoating the substrate bound nucleic acid precursor (see FIG. 2). The photoacid that is generated by the PPA technique then removes the acid labile dimethoxytrityl protecting group (DMT) and activates the surface bound linker molecule toward coupling the first nucleic acid base. The prior art process outlined in FIG. 2 can be repeated multiple times to construct arrays of oligonucleotides of the desired length.

Despite the above advances made in the area of biomolecular array fabrication, there is still a need for providing new and improved techniques for fabricating biomolecular arrays. In addition, there is a need for providing a technique for fabricating protein arrays, which cannot be fabricated using prior art techniques.

### SUMMARY OF THE INVENTION

The present invention provides a series of photoactivatible surface bound linker molecules which can be used to fabricate biomolecular arrays. Specifically, the present invention provides, in one embodiment, a composition which includes a solid substrate; an organic linking group having one terminal end portion bound to the solid substrate and at least one other terminal end portion containing an alcohol or carbonyl functionality; and an acid labile protecting group selected from acetals and ketals bound to the alcohol or carbonyl functionality. The present invention also contemplates a composition which includes a plurality of such surface bound linking groups which have alcohol or carbonyl functionality that is protected with an acetal or ketal.

The present invention also provides a composition which comprises a solid substrate; an organic linking group having one terminal end portion bound to the solid substrate and at least one other terminal end portion containing an aldehyde group. The present invention also contemplates a composition having a plurality of such organic linking groups bound to the surface of the solid substrate.

The present invention further provides a composition which includes a solid substrate; and at least one of a photoacid generator or a sensitizer bound to the solid substrate. The at least one photoacid generator or sensitizer bound composition may also include an acid labile protecting group selected from acetals and ketals bound to the photoacid generator or sensitizer. The photoacid generator or sensitizer may also include an organic linking group having one terminal end portion bound to the solid substrate and at least one other terminal end portion bound to the photoacid generator or sensitizer.

### BRIEF DESCRIPTION OF THE DRAWINGS

FIG. 1 is a schematic showing a prior art technique for nucleic acid synthesis.

FIG. 2 is a schematic showing a prior art technique referred to as Photoacid Patterned Array (PPA) for oligonucleotide synthesis.

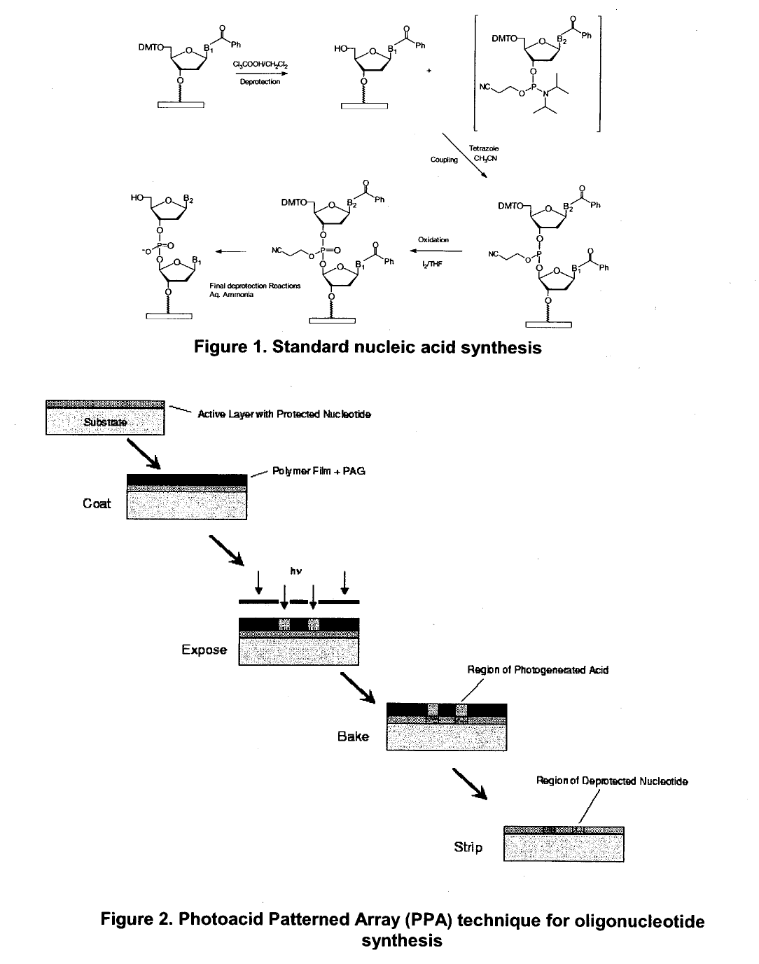

### DETAILED DESCRIPTION OF THE INVENTION

The present invention, which provides substrate bound linker molecules for the construction of biomolecule arrays and protein arrays, will now be described in greater detail.

One aspect of the present invention relates to a composition which includes a solid substrate; at least one organic linking group having one terminal end portion bound to the solid substrate and at least one other terminal end portion containing an alcohol or carbonyl functionality; and an acid labile protecting group selected from acetals and ketals bound to the alcohol or carbonyl functionality.

This aspect of the present invention provides improved acid labile protecting groups for oligonucleotide synthesis using conventional organic linking groups. The high reactivity (low activation energy) and efficient acid catalysis observed in the deprotection reaction of the inventive composition in this embodiment of the present invention is advantageous in array construction when compared to prior art dimethoxytrityl (DMT) protected alcohols due to higher synthesis yields and higher reactivity at low temperatures. Incorporation of acetal/ketal protected alcohols in the nucleotide building blocks is also possible.

One component of the inventive composition of the present invention is a solid substrate. Examples of solid substrates employed in the present invention include, but are not limited to: glass, doped glass, oxides such as silicon oxide, indium tin oxide (ITO) and titanium oxide, semiconductor substrates, metals such as Au, and the like. A preferred solid substrate employed in the present invention is a glass substrate.

The solid substrate is typically cleaned prior to use utilizing conventional cleaning techniques that are well known to those skilled in the art. For example, when the solid substrate is glass, the glass substrate is cleaned prior to linker attachment using various treatments such as, for example, sulfuric acid, deionized (DI) water, isopropylalcohol (IPA), heat, NaOH, oxygen plasma and hydrochloric acid. Mixtures of the aforementioned cleaning treatments are also contemplated in the present invention.

The solid substrate, which may optionally be cleaned prior to use, is typically wetted with an appropriate solvent prior to linker attachment. Suitable solvents that can be used to wet the surface of the solid substrate include, but are not limited to: alcohols, such as methanol, ethanol, butanol, pentanol, hexanol, heptanol, octanol, and nonanol; hydrocarbons such as pentane, hexane, and heptane; glycol ether acetates; glycol ethers; aromatic hydrocarbons such as toluene and xylene; chlorinated hydrocarbons such as choloroform and methylene chloride; and other like solvents. Of these solvents, alcohols such as ethanol are particularly preferred in the present invention for wetting the surface of the solid substrate.

The wetting of the surface of the solid substrate is typically performed at room temperature, but an elevated temperature up to, and including, the boiling point of the solvent may be employed. The duration of the wetting step may vary, but typically wetting is carried out for a time period of from about 1 to about 30 minutes, with a time period of from about 2 to about 10 minutes being more highly preferred.

Next, a solution comprising a ‘linker compound’ substantially dissolved in one of the above-mentioned solvents is applied to a surface of the solid substrate. The application of the ‘linker compound’ to the solid substrate is performed by conventional means well known to those skilled in the art. For example, the solid substrate can be immersed into the solution containing the ‘linker compound’ or the solution containing the ‘linker compound’ can be applied by brushing, dip coating, spraying or another like coating means.

The application of the solution containing the ‘linker compound’ to the solid substrate may be performed at room temperature or an elevated temperature up to, and including, the boiling point of the solvent may be employed. The duration of the ‘linker compound’ application step may vary depending on the surface area of the solid substrate, but typically the application of the ‘linker compound’ to the solid substrate is performed for a time period of from about 5 minutes to about 24 hours, with an application time period of from about 10 to about 30 minutes being more highly preferred.

The term ‘linker compound’ is used throughout the application to denote the reaction product that is formed between an organic linking group and a compound that is capable of bonding to the solid substrate. The solution employed at this point of the present invention typically contains from about 1 to about 20 wt. %, more preferably from about 2 to about 15 wt. %, and even more preferably from about 3 to about 10 wt. %, of linker compound dissolved in 100% solvent.

The compound which is capable of bonding to the solid substrate may be a bond, a carbon atom, a substituted carbon atom, a carboxylamide, a Si atom, a substituted Si atom such as trialkyloxysilyl, an ionic bond, a carboxylic acid, an amine, a thiol, a chlorodialkoxysilyl, an alkyldialkoxysilyl, or a dialkylalkoxysilyl. In the present invention, and when the solid substrate is comprised of glass, it is preferred that the terminal end portion of the linking compound that gets bound to the solid substrate includes a Si moiety. An example of a preferred linking group that may be employed in the present invention is an aminopropyltriethoxysilane.

The term “organic linking group” is used in the present invention to denote an organic compound including one terminal end portion that is capable of bonding to an organic compound which can be bound to the solid substrate, a bridging portion, and at least one other terminal end portion that includes an alcohol or carbonyl functionality. The organic linking group may be linear, polymeric or dendrimeric.

The bridging portion which links the terminal end portions together is a linear or branched, substituted or unsubstituted alkane, alkenyl, alkylcarboxylamide, aryl, alkoxylate, such as ethoxylate and propoxylate, aryloxylate or any combination thereof. The bridging portion of the organic linking group may contain from about 3 to about 600 carbon atoms, preferably from about 10 to about 200 carbon atoms, and more preferably from about 12 to about 100 carbon atoms. The substitute groups that may be present in the linking group include, but are not limited to: alkyls, alkenyls, halogens, ethers or amides.

In a preferred embodiment of the present invention, the bridging portion of the linking group is a dendrimeric compound, an alkane chain having the formula —CH₂)—ₙ wherein n is from about 3 to about 30, an ethoxylate having the formula —(CH₂CH₂O)—ₓ wherein x is from about 1 to about 50, and an aryloxylate having the formula —C₆H₄O)ₓ wherein x is from about 1 to about 50. The term “dendrimeric” is used in the present invention to denote a highly branched organic compound.

The acetals or ketals employed in the present invention include well known low-activation energy aliphatic or cyclic acetals or ketals. Some examples of acetals or ketals that can be employed in the present invention include, but are not limited to: dimethyl acetal or ketal, dioxolane, tetrahydrofuranyl, tetrahydropyranyl, methoxycyclohexanyl, methoxycyclopentanyl, cyclohexanyloxyethyl, ethoxycyclopentanyl, ethoxycyclohexanyl, methoxycycloheptanyl, and ethoxycycloheptanyl. Preferred acid labile ketals or acetals include: tetrahydropyranyl acetal, dimethyl acetal or ketal, or dioxolane.

Following the application of the linker compound to the solid substrate, the solid substrate containing bound linker is then rinsed with one of the above mentioned solvents and then the composition, i.e., substrate containing bound linker compound, is dried in air or under vacuum at room temperature or at an elevated temperature of up to, and including, 110° C. The drying step is performed for a time period of from about 5 to about 60 minutes, with a drying time of from about 10 to about 20 minutes being more highly preferred.

After drying, the composition including the bound linker compound is then baked in a furnace or on a hot plate. The temperature and duration of the baking step may vary depending on whether a hot plate or furnace is employed. When a hot plate is employed, the baking step is carried out at a temperature of from about 80° to about 150° C. for a time period of from about 5 to about 60 minutes. More preferably, the hot plate baking step is performed at a temperature of from about 100° to about 110° C. for a time period of from about 10 to about 20 minutes. When a furnace is employed in the baking step, the baking step is carried out at a temperature of from about 80° to about 150° C. for a time period of from about 5 to about 60 minutes. More preferably, the furnace baking step is performed at a temperature of from about 100° to about 110° C. for a time period of from about 10 to about 20 minutes.

Following the baking step, the attached linkers can now be deprotected in a patternwise fashion to provide reactive substrates for biomolecule arrays. The deprotection may be carried out by either heat or exposure to radiation using processes that are well known to those skilled in the art.

Illustrative examples of some of the preferred compositions of this embodiment of the present invention include the following:

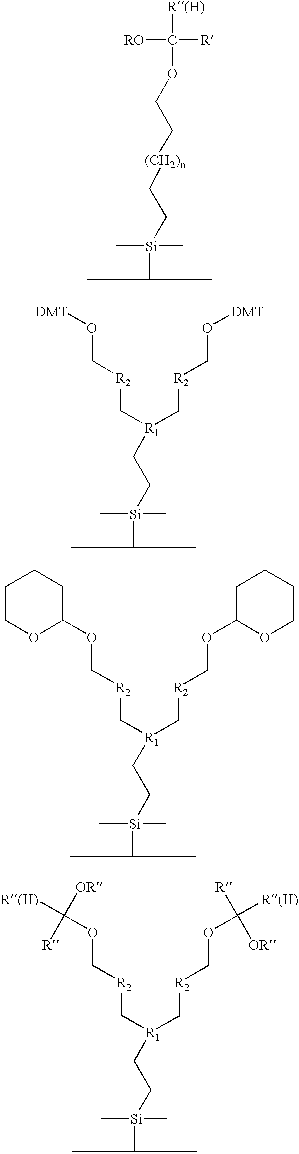

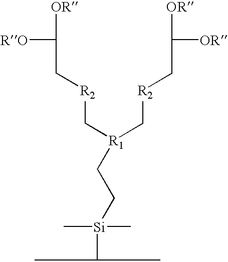

wherein R and R′ independently are an alkyl such as ethyl, methyl, etc., or R and R′ taken together form a cyclic alkane, R″ is H, an alkyl or R″ taken with either R or R′ forms a cyclic alkane, R₁ is a branching point such as a disubstituted acetamide or a trisubstituted aryl ether such as trioxysubstituted benzamide, R₂ is an alkyl, an alkoxy, an alkoxylate, an aryl or an aryloxylate; and n is from 0 to 20.

The substrate bound linkers with protected acid labile functionality described above can be used for oligonucleotide syntheses using standard nucleic acid synthesis or in forming biomolecular arrays. The substrate bound linkers with protected acid labile functionality may also be used for protein array fabrication. The preferred method for binding proteins to substrates in through the formation of Schiff bases with surface bound aldehydes. This can be accomplished with linkers incorporating acid labile acetals or ketals as described above by simply replacing the alcohol attachment site with aldehydes sites. This approach has the added advantage in that it is easy to interconvert the functionalized aldehyde and alcohol before attachment, thus a single silane precursor can be used for both DNA and protein chips.

The following scheme shows the substrate bound linkers with acid labile protection of aldehydes for protein attachment:

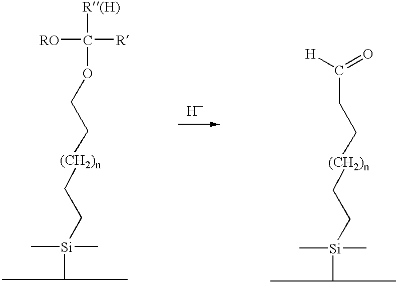

The present invention further provides a composition which includes a solid substrate; and at least one of a photoacid generator or a sensitizer bound to the solid substrate. The at least one photoacid generator or sensitizer bound composition may also include an acid labile protecting group selected from acetals and ketals bound to the photoacid generator or sensitizer. The photoacid generator or sensitizer may also include an organic linking group having one terminal end portion bound to the solid substrate and at least one other terminal end portion bound to the photoacid generator or sensitizer.

In this embodiment of the present invention, components of a polymer resist, i.e., photoacid generator or sensitizer are bound to the surface of the substrate as either part of the organic linker compound described above or as individual components.

In this embodiment, the compound containing linker and photoacid generator may be made as follows: A linker of a sulfonium salt PAG could be synthesized by the reaction of 3-bromopropyltrimethoxysilane or α-bromo-ω-trialkoxysilyl alkane with a diarylsulfide (phenyl sulfide) and then exchanging the bromo anion with the desired anion (i.e., triflate). Alternatively, the substrate bound photoacid generator could be made by linking the PAG via an aryl hydroxy group on the PAG to α-bromo-ω-vinylalkane followed by treatment with a trialkoxysilane.

Suitable photoacid generators (PAGs) that may be employed in the present invention include: triflates (e.g., triphenylsulfonium triflate or bis-(t-butylphenyl) iodonium triflate), pyrogallol (e.g., trimesylate or pyrogallol), onium salts such as a triarylsulfonium and diaryl iodonium hexafluoroantimates, hexafluoroarsenates, trifluoromethane sulfonates and others; iodonium sulfonates and trifluoromethanesulfonate esters of hydroxyamines, alpha′-bis-sulfonyl diazomethanes, sulfonate esters of nitro-substituted benzyl alcohols and napthoquinone-4-diazides and alkyl disulfonates. Other suitable photoacid generators for use in the present invention are disclosed, for example, in U.S. Pat. Nos. 5,045,431 and 5,071,730 both to Allen, et al. and the Reichmanis, et al. review article (Chemistry of Materials, Vol. 3, page 395 (1991)), the disclosures of which are incorporated herein by reference.

A linker with a sensitizer could be prepared by the reaction of α-bromo-ω-vinylalkane with a hydroxyl substituted sensitizer such as 9-anthracene methanol, followed by treatment with a trialkoxysilane.

Examples of sensitizers, i.e., photosensitizers, that may be employed in the present invention include: chrysenes, pyrenes, fluoranthenes, anthrones, benzophenones, thioxanthones, and anthracenes, such as 9-anthracene methanol (9-AM). Additional anthracene derivative sensitizers are disclosed in U.S. Pat. No. 4,371,605. The sensitizer may include oxygen or sulfur.

The following depicts some of the compositions of the present invention that include a sensitizer with and without an acid labile acetal or ketal protecting group, and a photoacid generator with and without an acid labile acetal or ketal protecting group.

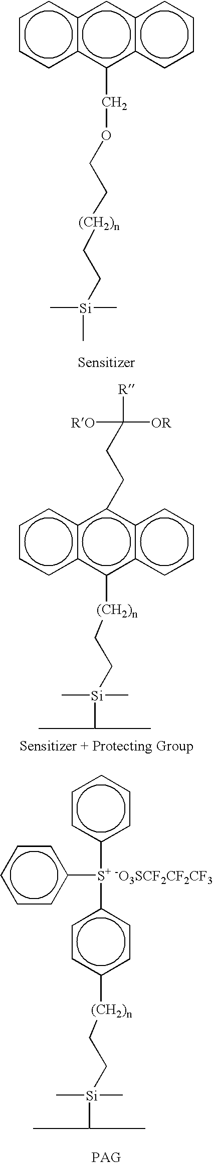

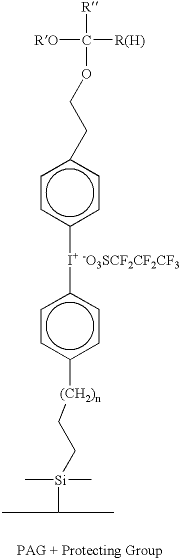

The advantages found in employing the substrate bound photoacid generators and sensitizers include the following:

(1) Potential for use in an “all dry” format : Because the photoacid generator (and sensitizer) is bound to the substrate there is in principle no need for an overcoat polymer resist or other solvent provided the monomolecular layer of linker compounds provides adequate solvation for the photochemical and thermal reactions that occur during exposure and deprotection. The polyether dendrimers are expected to be particularly useful in this regard. The catalytic nature of the deprotection reactions and the high concentrations of acid that could be produced favor the use of relatively low amounts of substrate bound PAGs. Diffusion of the photogenerated acid could still be an issue and may have to be controlled. Subsequent oligonucleotide and protein coupling reactions can be performed in the usual manner. The absence of a polymer coating step greatly simplifies array fabrication.

(2) In the event that the “all dry” approach is not feasible, substrate bound PAGs and sensitizers could be used in a solvent based approach as well. Here problems with acid diffusion would be minimized (lower acid concentrations are required and they are generated at the surface). This is true even if one of the components is in solution. Solvent based approaches could be used either with high resolution oligonucleotide arrays or preferably with low density arrays employing direct write schemes.

(3) Multiple exposures and sequential reactions using substrate bound PAGs would rely on the catalytic nature of any deprotection reaction utilized. Therefore, judicious control of substrate bound PAG would ensure that only partial use of the PAG occurs at any step and allowing subsequent exposures to catalyze further deprotection chemistry.

The following examples are provided to illustrate the method of the present invention which is employed in forming photochemically active surface bound linker molecules which can be used to fabricate bimolecular arrays.

### EXAMPLE 1

### Synthesis of Linker with Acetal Protected Aldehyde Group for Protein Array Fabrication

In this example, the spacer between the silica reactive group and the bio-reactive group is an alkane chain (CH₂)₁₂.

12-Hydroxydodecanoic acid (2.15 g, 9.93 mmol) was converted to the methyl ester by treatment with 1.34 mL of 1N hydrochloric acid in 160 ml methanol at room temperature overnight. After neutralization with dilute sodium hydroxide, the solvent was removed by rotary evaporation and the product was redissolved in ethyl acetate, washed with deionized (DI) water, and 5% sodium bicarbonate, dried over sodium sulfate and rotary evaporated to dryness yielding 2.00 g, 8.68 mmol (87.4% crude yield) of methyl 12-hydroxydodecanoate.

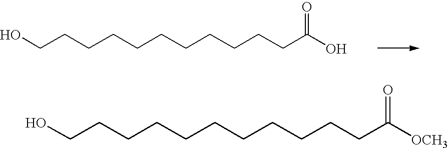

The aldehyde was prepared as described by J. E. Baldwin, R. M. Adlington, and S. H. Ramcharitar, Tetrahedron 1992, 48, 2963. Oxalyl chloride (1.27 g, 9.98 mmol) and 20 mL methylene chloride were placed in a 3 necked round bottom flask equipped with 3 addition funnels, one containing 1.5 mL DMSO in 4 mL methylene chloride, the second containing 2.00 g methyl 12-hydroxydodecanoate in 5 mL methylene chloride, and the third with 6 mL triethylamine. After cooling the flask to −50° C., the DMSO solution was added dropwise rapidly. After 10 minutes, the alcohol was added dropwise over 10 minutes. After 45 minutes the triethylamine was added dropwise. After stirring a further 10 minutes, the flask was allowed to warm to room temperature and stir overnight. 50 mL methlene chloride was added and the solution was washed with water (3×50 mL), dried over magnesium sulfate, filtered, and rotary evaporated to dryness yielding 2.03 g, 8.85 mmol of methyl 12-oxadodecanoate.

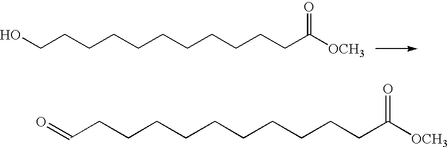

The aldehyde group was protected as described by V. Pozsgay, J. Org. Chem 1998, 63, 5998. The methyl 12-oxadodecanoate (2.02 g, 8.85 mmol) was dissolved in 15 mL 2,2-dimethoxypropane and p-toluenesulfonic acid monohydrate (0.1 7 g, 0.885 mmol) was added. After stirring for 30 minutes, the volume was reduced by about ½ by rotary evaporation, treated with excess triethylamine, diluted with methylene chloride, washed with water, dried over sodium sulfate and rotary evaporated to dryness yielding 2.11 g, 7.69 mmol of methyl 12,12-dimethoxydodecanoate. This was redissolved in 15 mL methanol and 11.4 mL 1N NaOH was added. After 45 minutes, the methanol was removed by rotary evaporation. The solution was washed with ether three times. Solid citric acid was added to the aqueous solution until a pH of 3.5 was obtained. The product was extracted into methylene chloride (3×) and the combined organic phase was washed with water, dried over magnesium sulfate, concentrated by rotary evaporation, dried in a vacuum oven to 1.95 g pale yellow oil, 7.49 mmol of 12,12-dimethoxydodecanoic acid.

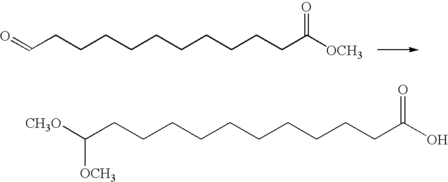

3-aminopropyltriethoxysilane (APTES) (1.66 g, 7.49 mmol), 12,12-dimethoxydodecanoic acid (1.95 g, 7.49 mmol), and DCC (1.85 g, 8.99 mmol) were combined in 100 mL of 1:1 ethylacetate/methylene chloride solvent mixture and allowed to stir overnight. The solids were filtered off and the filtrate was rotary evaporated to an 4.33 g of 12,12-dimethoxy-N-(3′-triethoxysilylpropyl)dodecanamide, a linker with an acetal protected aldehyde group for protein array fabrication.

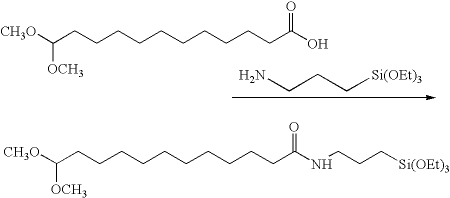

### EXAMPLE 2

### Synthesis of Linker with Acetal Protected Aldehyde Group for Protein Array Fabrication

In this example, the spacer between the silica reactive group and the bio-reactive group is polyethyleneglycol (PEG) which can provide an inert surface for biomolecules. PEG has two symmetric (hydroxyl) end groups. This has to be functionalized unsymmetrically with the protected bio-reactive group on one end and the silica reactive group on the other by doing a partial functionalization and separation/isolation of the unsymmetrically substituted polymer.

As described by A. Dal Pozzo, A. Vigo, and G. Donzelli in Makromol. Chem. 1989, 190, 2457-2461, PEG of molecular weight of 600 (21.70 g, 36.17 mmol) was treated with trityl chloride (10.08 g, 36.17 mmol) and 5 ml triethylamine (36.17 mmol) in 90 mL methylene chloride and stirred at room temperature for 2 hours. The slurry was washed with DI water (1×180 mL, 5×90 mL) to remove the unreacted PEG, rotary evaporated to dryness. The oil was redissolved in toluene (100 mL) and washed with brine (1×50 mL, 5×25 mL), dried over sodium sulfate, filtered, rotary evaporated, and dried in a vacuum oven to yield 22.98 g of oil which is a mixture of bis-tritylated PEG and mono-tritylated PEG.

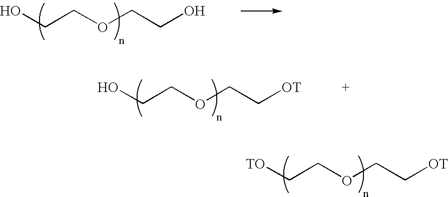

As described by T. Bigo, N. D. Sachinvala, and O. A. Hamed in Polymer Prepr. 2000, 41(1), 144, NaH (5.73 g, 143.16 mmol) was washed with hexanes, slurried in 100 mL THF, and taken to reflux. The mixture of tritylated PEG dissolved in 60 mL THF was added dropwise over ½ hour and stirred at reflux for 4½ hours. Chloroacetic acid (2.25 g, 23.86 mmol) dissolved in 60 mL THF was added dropwise over ½ hour. The slurry was stirred at reflux overnight, cooled to room temperature and 60 mL water was added. The pH was adjusted to ˜5 with dilute hydrochloric acid and the volatiles were removed by rotary evaporation. The slurry was triturated with ether (3×60 mL) to remove bis-tritylated PEG and chloroacetic acid. The slurry was redissolved in methylene chloride, dried over magnesium sulfate, filtered, rotary evaporated, and dried in a vacuum oven to 11.62 g (12.91 mmol) of α-trityl-ω-carboxy-PEG.

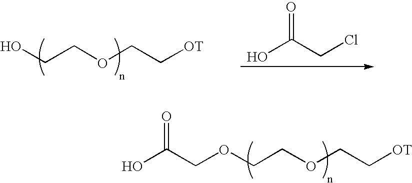

The α-trityl-ω-carboxy-PEG was treated with 2 mL of 12 N hydrochloric acid in 260 mL methanol at room temperature overnight. After neutralization with solid sodium carbonate, the solution was filtered and rotary evaporated, redissolved in brine, and washed with ether (5×100 mL). The aqueous solution was neutralized with hydrochloric acid, and the product was extracted into methylene chloride (4×200 mL), dried over magnesium sulfate and rotary evaporated to an oil (7.04 g, 10.49 mmol) of α-hydroxy-ω-carboxy-PEG.

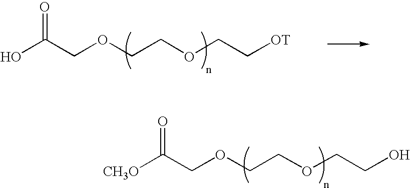

The α-hydroxy-ω-methylcarboxy-PEG was converted to an aldehyde, protected, and further reacted with APTES as in Example 1 to prepare α,α-dimethoxy-ω-[N-(3′-triethoxysilylpropyl) carboxamide-PEG, a linker with an acetal protected aldehyde group for protein array fabrication.

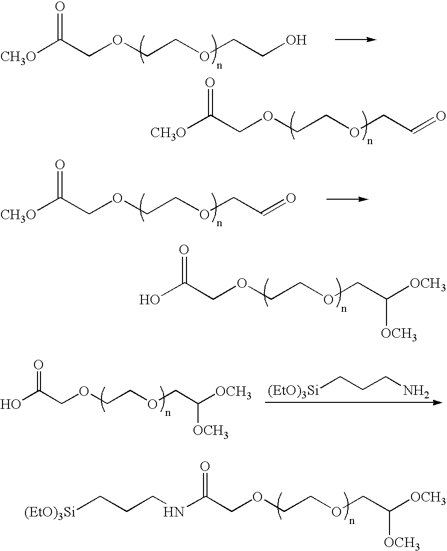

Alternatively, the protected aldehyde functionality can be added in one step by treating the α-hydroxy-ω-methylcarboxy-PEG (8.23 mmol) with sodium hydride (2.00 g, 49.38 mmol) and bromoacetaldehyde dimethyl acetal (2.78 g, 16.47 mmol) in refluxing THF overnight. After the addition of water, the solvents are removed by rotary evaporation. Ether trituration removes excess bromoacetaldehyde. The slurry is made acidic by the addition of solid citric acid, and the product is extracted into methylene chloride, washed with water, dried over magnesium sulfate, filtered and rotary evaporated to α,α-dimethoxy-ω-carboxy-PEG which can be reacted with APTES as above to prepare the desired linker.

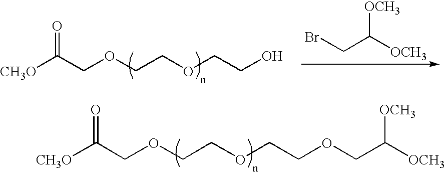

### EXAMPLE 3

### Synthesis of Linker with Cyclic Acetal Protected Alcohol

The α-hydroxy-ω-methylcarboxy-PEG (from example 2) (3 mmol) was reacted with sodium hydride (1.44 g, 36 mmol) and 2-(2-chloroethoxy)tetrahydro-2H-pyran (0.99 g, 6.01 mmol) in refluxing THF overnight. After the addition of water, the solvents are removed by rotary evaporation. Ether trituration removes excess 2-(2-chloroethoxy)tetrahydro-2H-pyran The slurry is made acidic by the addition of solid citric acid, and the product is extracted into methylene chloride, washed with water, dried over magnesium sulfate, filtered and rotary evaporated to α,α-dimethoxy-ω-methylcarboxy-PEG which can be reacted with APTES as above to prepare the desired linker.

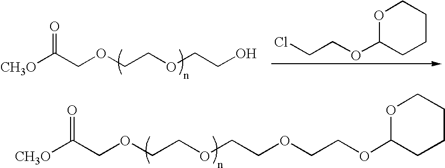

### EXAMPLE 4

### Synthesis of Linker with DMT Protected Alcohol

The α-hydroxy-ω-methylcarboxy-PEG (from example 2) was hydrolysed by acid treatment and the resulting α-hydroxy-ω-carboxy-PEG (3.04 g, 4.62 mmol) was treated with dimethoxytritylchloride (DMTCl) (3.13 g, 9.24 mmol) in 100 mL 1:1 pyridine/methylene chloride at room temperature overnight. The solvent was removed by rotary evaporation and the product was redissolved in ether, filtered to remove salts, and rotary evaporated to dryness. The α-DMToxy-ω-carboxy-PEG was then reacted with APTES as in Example 1 to yield α-DMToxy-ω-[N-(3′-triethoxysilylpropyl) carboxamide-PEG, a linker with a DMT protected alcohol for bio-molecule array fabrication.

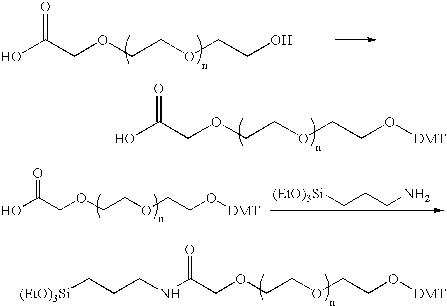

### EXAMPLE 5

### Synthesis of 2-Branched Linker for Biomolecule Attachment

The mono-tritylated PEG is then reacted with dichloroacetic acid instead of chloroacetic acid to give a branched linker which is separated and purified as described in Example 2.

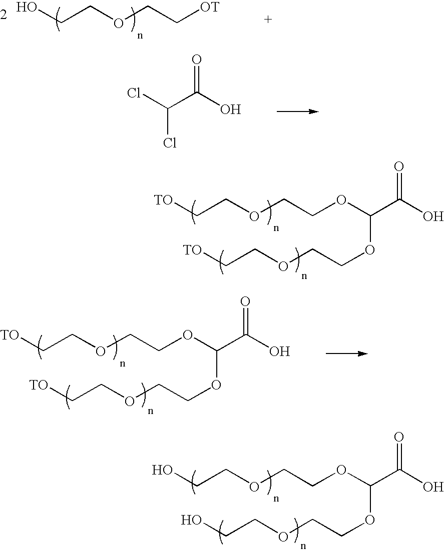

The branched linker can then be modified and protected as desired (P) and then reacted with APTES to give a functional branched linker.

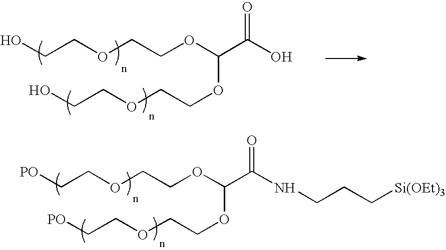

### EXAMPLE 6

### Synthesis of 3-Branched Linker for Biomolecule Attachment

As described in S. J. Meunier, Q. Wu, S.-N. Wang, and R. Roy, Can. J Chem. 1997, 75, 1472-1482, p-toluenesulfonyl chloride (6.88 g, 36 mmol) was added slowly under nitrogen to a 0° C. solution of the mono-tritylated PEG from Example 2 (33.3 mmol) and 50 mL triethylamine diethyl ether (300 mL). The slurry was allowed to warm to room temperature and stirred overnight. The solvent was rotary evaporated and the residue was dissolved in methylene chloride and washed with saturated sodium bicarbonate solution (50 mL) and water (2×50 mL). The organic layer was dried over magnesium sulfate and rotary evaporated to dryness to α-trityloxy-ω-p-toluenesulfonyloxy-PEG

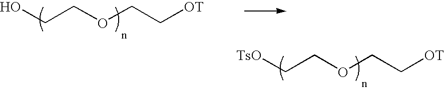

This was dissolved in DMF (250 mL) along with ethyl gallate (1.65 g, 8.325 mmol) and potassium carbonate (11.65 g, 83.25 mmol) and stirred at 80° C. for 2 days. After cooling to room temperature, the solids were filtered off, the solvents were removed under vacuum, and then coevaporated with t-butanol to help remove the DMF. Water was added and the product was extracted into methylene chloride. The organic layer was washed with water and brine, dried over magnesium sulfate, filtered, and rotary evaporated to a brown oil.

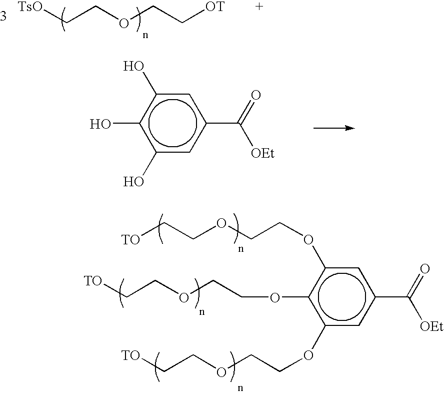

The ethyl 3,4,5-tri-(ω-trityloxy-PEG)benzoate was stirred with 1.5 N sodium hydroxide (200 mL) in methanol (200 mL) overnight at room temperature. The solvents were removed by rotary evaporation and the residue was triturated with ether (3×100 mL) to remove the bis-tritylated PEG. The residue was then dissolved in methylene chloride, dried over magnesium sulfate and rotary evaporated to 22.35 g of 3,4,5-tri-(ω-trityloxy-PEG)benzoic acid. After stirring at room temperature overnight with 12 N hydrochloric acid (4 mL) in 250 mL methanol, neutralization with solid sodium carbonate, ether washing to remove trityl alcohol and extraction into methylene chloride, a trifunctional linker was prepared which could be reacted/protected at the hydroxyl ends followed by reaction with APTES to prepare biofunctional/silica reactive linkers.

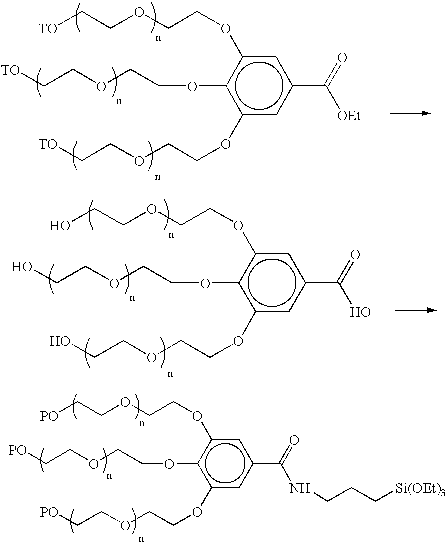

### EXAMPLE 7

### Attachment of Linkers to Silica Substrates

The substrates are cleaned immediately prior to linker attachment in order to ensure the presence of reactive hydroxyl groups on the surfaces. This may include treatments with sulfuric acid, DI water, IPA, heat, NaOH, and hydrochloric acid. The substrates are then soaked in ethanol for 5 minutes, followed by a 15 minute soak in a 5% solution of the linker(s) dissolved in ethanol, followed by another minute soak in ethanol. After drying in a stream of air, the substrates are baked for 15 minutes at 110° C. resulting in substrates with the desired linker(s) attached which can now be deprotected in a patternwise fashion to provide reactive substrates for biomolecule arrays.

### EXAMPLE 8

A variety of branched polyhydroxy acids and esters can be prepared as described in Scheme 1 using orthogonal protecting groups. In this case, benzyl esters were prepared from the carboxylic acids and the hydroxy groups were protected as the t-butyldimethylsilyl ethers. The esters could be selectively cleaved by catalytic hydrogenolysis and the silyl groups removed with boron trifluoride etherate. Each of these processes could be accomplished selectively. The starting material for the synthesis was Bis-(hydroxymethyl) propionic acid (DMPA). Using the chemistry and the simple building blocks described in Scheme 1, a variety of polyhydroxyl acid and ester derivatives containing 2, 4, 8, 16 and more pendant hydroxyl functionalities could be prepared. Using selective protection/deprotection schemes, these materials could be attached to linkers such as APTES and others as described. In addition, aliphatic ester spacers could be added using the hydroxy functionality to initiate ring opening polymerizations of various lactones including caprolactone. These polymerizations are living using various metal and organic catalysts and result in branched polyesters of various architectures with hydroxyl termination. The incorporation of spacers provides additional separation between the terminal functionality. (M. Trollsas, J. L. Hedrick J. Am. Chem. Soc. 1998, 120, 4644; M. Trollsas, H. Claesson, B. Atthoff, J. L. Hedrick Angew. Chem. Int. Ed. 1998,37(22), 3132; M. Trollsas, J. L. Hedrick, D. Mecerreyes, Ph. Dubois, R. Jerome, H. Ihre, A. Hult Macromolecules, 1998, 31, 2756).

2,2-Bis(tert-butyldimethylsiloxymethyl) Benzyl Propionate G1 (CO2Bz, TBDMS):

2,2 Bis(hydroxymethyl) benzyl propionate (49.8 g, 222 mmol), tert-butyldimethylsilyl chloride (TBDMSCl) (80.5 g, 535 mmol) and imidazole (37.8 g, 533 mmol) were dissolved in 150 mL of methylene chloride. The mixture was stirred for 12 h at room temperature and the solvent evaporated. The crude residue was dissolved in hexane and extracted with water. The organic phase was evaporated to yield 95.2 g (94%) of a colorless liquid. ¹H-NMR(CDCl₃) δ: 0.00 (s, 12H), 0.83 (s, 18H), 1.12 (s, 3H), 3.64 -3.77 (q, 4H), 5.10 (s, 2H), 7.32 (s, 5H).

General Procedure for Desilylation:

Into a flask under nitrogen was placed 0.52 mmol of TBDMS- protected material, 30 mL of dry methylene chloride and BF₃-Et₂O (92.6 mmol). The mixture was stirred for 12 h at 40° C. and poured into cold methanol. The product was isolated by decantation or filtration and used without further purification.

2,2-Bis(tert-butyldimethylsiloxymethyl) Propionic Acid G1(CO2H, TBDMS) and a General Procedure for Removal of the Benzyl Ester Groups:

G1 (CO2Bz, TBDMS) (210 mmol, 95.2 g) was dissolved in EtOAc (100 mL) and Pd/C (10 wt %)(1.5 g) was added. The hydrogenation bottle was filled with hydrogen (50 psi) and shaken for 6 h at room temperature. The reaction was stopped and the catalyst filtered. The solvent was evaporated to yield 94.2 g, (99%) of a colorless liquid. ¹H-NMR (CDCl₃) δ: 0.00 (s, 12H), 0.82 (s, 18H), 1.07 (s, 3H), 3.6-3.69 (q, 4H).

G2(CO2Bz, TBDMS) and General Procedure for Esterification Using DCC:

2,2-Bis(hydroxymethyl) benzyl propionate (23.3 g, 104 mmol), G1(CO2H, TBDMS) (79 g, 218 mmol) and 4-(dimethylamino) pyridium p-toluenesulfonate (DPTS) were dissolved in 150 mL of methylene chloride. Dicyclohexylcarbodiimide (DCC) (55.7 g, 270 mmol) was then added and the reaction stirred at room temperature for 12 h. The mixture was filtered and the filtrate purified by column chromatography ( silica gel, hexane, EtOAc 95:5 eluant). The yield was 30 g (32%) of a colorless, viscous oil: ¹H-NMR(CDCl₃) δ: 0.00 (s, 24H), 0.84 (s, 36H), 1.09 (s, 6H), 1.23 (s, 3H), 3.56-3.70 (q, 8H), 4.15-4.30 (q, 4H), 5.13 (s, 2H), 7.33 (s, 5H).

G2(CO2H, TBDMS):

G2(CO2Bz, TBDMS) (30.0 g, 32.9 mmol) and Pd/C (1.5 g) were dissolved in 100 mL of EtOAc and treated with hydrogen for 4h as described above. The yield was 26.2 g (97%) of a colorless, viscous liquid. ¹H-NMR (CDCl₃) δ: 0.00 (s, 24H), 0.84 (s, 36H), 1.06 (s, 6H), 1.25 (s, 3H), 3.57-3.72 (q, 8H), 4.06-4.29 (m, 4H), 5.28 (s, 2H).

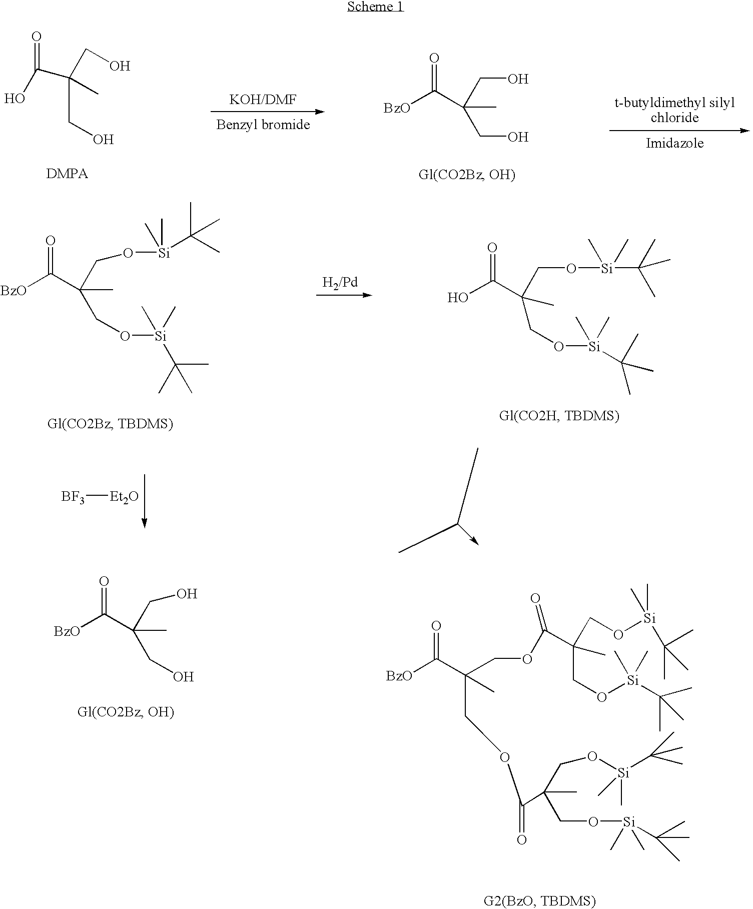

While the present invention has been particularly shown and described with reference to preferred embodiments thereof, it will be understood by those skilled in the art that various changes in forms and details may be made herein without departing from the spirit and scope of the present invention. It is therefore intended that the present invention is not limited to the exact forms and details described and illustrated, but fall within the scope of the appended claims.

We claim:

1. A composition comprising a solid substrate; an organic linking group having one terminal end portion bound to the solid substrate and at least one other terminal end portion containing an alcohol or carbonyl functionality; and an acid labile protecting group selected from the group consisting of acetals and ketals bound to the alcohol or carbonyl functionality.

2. The composition of claim 1 wherein said solid substrate is selected from the group consisting of glass, doped glass, an oxide, a semiconductor and a metal.

3. The composition of claim 1 wherein said solid substrate is glass.

4. The composition of claim 1 further comprising a plurality of said organic linking groups that are protected with said acetal or ketal.

5. The composition of claim 1 wherein said organic linking group is selected from the group consisting of a linear linking group, a polymeric linking group and a dendrimeric linking group.

6. The composition of claim 1 wherein said organic linking group includes a bridging group between said terminal end portions.

7. The composition of claim 6 wherein said bridging group is an alkane chain having the formula —(CH₂)—ₙ wherein n is from about 3 to about 30.

8. The composition of claim 6 wherein said bridging group is an ethoxylate having the formula —(CH₂CH₂O)—ₓ wherein x is from about 1 to about 50.

9. The composition of claim 1 wherein said terminal end portion bound to said solid substrate comprises a substituted Si atom.

10. The composition of claim 1 wherein said acetal or ketal is an aliphatic or cyclic compound.

11. The composition of claim 1 wherein said acetal or ketal is selected from the group consisting of dimethyl acetal or ketal, dioxolane, tetrahydrofuranyl, tetrahydropyranyl, methoxycyclohexanyl, methoxycyclopentanyl, cyclohexanyloxyethyl, ethoxycyclopentanyl, ethoxycyclohexanyl, methoxycycloheptanyl, and ethoxycycloheptanyl.

12. The composition of claim 1 wherein said acetal or ketal is selected from the group consisting of tetrahydropyranyl acetal, dimethyl acetal or ketal, and dioxolane.

13. The composition of claim 1 wherein said acetal or ketal is deprotected by heat or exposure to radiation.

14. A composition comprising a solid substrate; an organic linking group having one terminal end portion bound to the solid substrate; and at least one other terminal end portion containing an aldehyde group.

15. A composition comprising a solid substrate and at least one of a photoacid generator or a sensitizer bound to the solid substrate.

16. The composition of claim 15 further comprising an acid labile protecting group selected from the group consisting of acetals and ketals bound to the photoacid generator or sensitizer.

17. The composition of claim 15 further comprising an organic linking group having one terminal end portion bound to the solid substrate and at least one other terminal end portion bound to the photoacid generator or sensitizer.

18. The composition of claim 15 wherein said solid substrate is selected from the group consisting of glass, doped glass, an oxide, a semiconductor, and a metal.

19. The composition of claim 15 wherein said solid substrate is glass.

20. The composition of claim 17 wherein said organic linking group is selected from the group consisting of a linear linking group, a polymeric linking group and a dendrimeric linking group.

21. The composition of claim 17 wherein said organic linking group includes a bridging group between said terminal end portions.

22. The composition of claim 21 wherein said bridging group is an alkane chain having the formula —(CH₂)—ₙ wherein n is from about 3 to about 30.

23. The composition of claim 21 wherein said bridging group is an ethoxylate having the formula —(CH₂CH₂O)—ₓ wherein x is from about 1 to about 50.

24. The composition of claim 16 wherein said acetal or ketal is an aliphatic or cyclic compound.

25. The composition of claim 16 wherein said acetal or ketal is selected from the group consisting of dimethyl acetal or ketal, dioxolane, tetrahydrofuranyl, tetrahydropyranyl, methoxycyclohexanyl, methoxycyclopentanyl, cyclohexanyloxyethyl, ethoxycyclopentanyl, ethoxycyclohexanyl, methoxycycloheptanyl, and ethoxycycloheptanyl.

26. The composition of claim 16 wherein said acetal or ketal is selected from the group consisting of tetrahydropyranyl acetal, dimethyl acetal or ketal, and dioxolane.

27. The composition of claim 16 wherein said acetal or ketal is deprotected by heat or exposure to radiation.

28. The composition of claim 15 wherein said photoacid generator is selected from the group consisting of triflates, pyrogallols, onium salts, iodonium sulfonates, trifluoromethanesulfonate esters of hydroxyamines, alpha′-bis-sulfonyl diazomethanes, sulfonate esters of nitro-substituted benzyl alcohols and napthoquinone-4-diazides and alkyl disulfonates.

29. The composition of claim 15 wherein said photoacid generator is selected from the group consisting of a triflate and an onium salt.

30. The composition of claim 15 wherein said sensitizer is selected from the group consisting of chrysenes, pyrenes, fluoranthenes, anthrones, benzophenones, thioxanthones, and anthracenes.

31. A method for forming a photoactivatible surface bound linker molecule comprising:
applying a solution comprising a linker compound to a wetted surface of a solid substrate, said linker compound comprising a component that bonds to said solid substrate;
drying the solid substrate containing the bound linker compound; and
baking the dried solid substrate.

32. The method of claim 31 further comprising cleaning said solid support prior to said applying.

33. The method of claim 32 wherein said cleaning is selected from the group consisting of sulfuric acid treatment, deionized water treatment, isopropylalcohol treatment, heat treatment, NaOH treatment, oxygen plasma treatment and hydrochloric acid treatment.

34. The method of claim 31 wherein said wetted solid substrate is formed by applying a solvent to said solid substrate, said solvent is selected from the group consisting of alcohols, hydrocarbons, glycol ether acetates, glycol ethers, aromatic hydrocarbons, and chlorinated hydrocarbons.

35. The method of claim 34 wherein said wetting is performed at room temperature up to the solvent's boiling point.

36. The method of claim 31 wherein said solution containing said linker compound contains from about 1 to about 20 wt. % linker compound dissolved in solvent.

37. The method of claim 31 wherein said linker compound is a reaction product formed by reacting an organic linking group and a compound that is capable of bonding to the solid substrate.

38. The method of claim 31 further comprising deprotecting said linker compound.

39. The method of claim 38 wherein said deprotecting is by heat or by exposure to radiation.
